(19) United States
(12) Patent Application Publication
(10) Pub. No.: US 2016/0201121 A1
(43) Pub. Date: Jul. 14, 2016

(54) MICROVESICLE-BASED ASSAYS

(71) Applicant: THE GENERAL HOSPITAL CORPORATION, Boston, MA (US)

(72) Inventors: Walter Chen, Cambridge, MA (US); Xandra O. Breakefield, Newton, MA (US); Leonora Balaj, Charlestown, MA (US); Johan Karl Olov Skog, New York, NY (US)

(73) Assignee: THE GENERAL HOSPITAL CORPORATION, Boston, MA (US)

(21) Appl. No.: 14/981,062

(22) Filed: Dec. 28, 2015

Related U.S. Application Data
(63) Continuation of application No. 13/878,668, filed on Oct. 17, 2013, now abandoned.
(60) Provisional application No. 61/393,600, filed on Oct. 15, 2010.

(51) Int. Cl. C12Q 1/68

(52) U.S. Cl. CPC C12Q 1/6827; C12Q 1/6886; C12Q 2600/118; C12Q 2600/156

(57) ABSTRACT

Methods are disclosed herein for assaying a biological sample or a bodily fluid obtained from a subject by isolating, obtaining or using a microvesicle fraction from the biological sample or bodily fluid and detecting in the microvesicle fraction the presence or absence of a genetic aberration in an IDH1, TDH2, TP53, PTEN, CDKN2A, NF1, EGFR, RB1, PIK3CA, or BRAF gene. The methods may be used for aiding the diagnosis, prognosis, monitoring, or therapy selection in relation to a disease or other medical condition (e.g., a glioma) in a subject.

### CROSS REFERENCE TO RELATED APPLICATIONS

This application claims the benefit of 35 U.S.C. §119(e) to U.S. Provisional Patent Application No. 61/393,600, filed Oct. 15, 2010, which is incorporated herein by reference in its entirety.

### FIELD OF THE INVENTION

The present invention relates to molecular diagnostics, particularly in the fields of medical diagnosis, prognosis, patient monitoring, and treatment efficacy based on the analysis of nucleic acids extracted from microvesicles.

### BACKGROUND

Molecular diagnostics, used to diagnose, monitor, treat, and evaluate diseases and other medical conditions, is becoming an increasingly important tool, particularly with the accumulating knowledge of the molecular mechanisms underlying various types of diseases and medical conditions. Molecular diagnostics is particularly valuable in the context of cancer, since our knowledge of the underlying genetic causes of cancers is rapidly expanding.

Cancers arise through accumulation of genetic alterations that promote unrestricted cell growth. It has been stated that each tumor harbors, on average, around 50-80 mutations that are absent in non-tumor cells (Jones et al., 2008; Parsons et al., 2008; Wood et al., 2007).

Current technologies to detect genetic mutations include the analysis of biopsy samples and the non-invasive analysis of mutant tumor DNA fragments circulating in bodily fluids, such as blood (Diehl et al., 2008). The former method is invasive, complicated, possibly harmful to subjects, and not particularly sensitive. The latter method inherently lacks sensitivity due to the extremely low copy number of mutant cancer DNA in bodily fluid (Gormally et al., 2007). Therefore, one challenge facing cancer diagnosis is to develop a diagnostic method that can detect tumor cells at different stages non-invasively, yet with high sensitivity and specificity.

The present invention discloses novel methods of detecting genetic aberrations within a microvesicle fraction isolated from a biological sample. The methods may be used for the diagnosis, prognosis and monitoring of a disease or other medical condition in a subject, or for selecting promising, optimal or individualized therapies for a disease or other medical condition in a subject.

### BRIEF SUMMARY OF THE INVENTION

One aspect of the invention are methods for assaying a biological sample, comprising the steps of (a) isolating, obtaining or using a microvesicle fraction from a biological sample; and (b) detecting in the microvesicle fraction the presence or absence of a genetic aberration in a gene selected from the group consisting of IDH1, IDH2, TP53, PTEN, CDKN2A, NF1, EGFR, RB1, PIK3CA, and BRAF. In certain of these methods, the genetic aberration is the G295A mutation in the IDH1 gene. In certain of the foregoing methods, the disease is cancer, for example, but not limit to glioma (e.g., but not limited to astrocytomas, oligodendrogliomas, oligoastrocytomas, or secondary glioblastomas), leukemia, or melanoma. In certain of the foregoing methods, the biological sample is a bodily fluid such as, but not limited to blood, plasma, serum, urine, or combinations thereof. In certain of the foregoing methods, the biological sample is from a human.

A further aspect of the invention are methods for aiding in diagnosis, prognosis, monitoring, or therapy selection in relation to a disease or other medical condition in a subject (for example, but not limited to, a human) comprising the steps of (a) isolating, obtaining or using a microvesicle fraction from a bodily fluid from a subject; and (b) detecting in the microvesicle fraction the presence or absence of a genetic aberration in a gene selected from the group consisting of IDH1, IDH2, TP53, PTEN, CDKN2A, NF1, EGFR, RB1, PIK3CA, and BRAF, wherein the genetic aberration is associated with the diagnosis, prognosis, monitoring, or therapy selection in relation to a disease or other medical condition. In certain of these methods, the genetic aberration is the G295A mutation in the IDH1 gene. In certain of these methods, the disease is cancer, for example, but not limit to glioma (e.g., but not limited to astrocytomas, oligodendrogliomas, oligoastrocytomas, or secondary glioblastomas), leukemia, or melanoma. In certain of the foregoing methods, the bodily fluid includes, but is not limited to blood, plasma, serum, urine, or combinations thereof.

In any of the foregoing aspects of the invention, the methods may further comprise (1) a step of extracting nucleic acids from the microvesicle fraction prior to detection of the genetic aberration; and/or (2) a step of treating the microvesicle fraction with DNase, RNAse inhibitor, or a combination of DNase and RNase inhibitor prior to or together with the step of extracting nucleic acids from the microvesicle fraction. In any of the aspects of the invention involving the step of extracting nucleic acids from the microvesicle fraction prior to detection of the genetic aberration, the extracted nucleic may be RNA, which, in turn, may be reverse-transcribed into complementary DNA.

In any of the foregoing aspects of the invention, the nucleic acid may be amplified prior to analysis, and said amplification may be carried out by polymerase chain reaction (PCR) or any of its variants such as in situ PCR, quantitative PCR, nested PCR; self-sustained sequence replication or any of its variants; transcriptional amplification system or any of its variants; Qb Replicase or any of its variants; or cold-PCR.

In yet another aspect of the invention, the detection of the presence or absence of a genetic aberration is performed using a digital PCR method, for example, but not limited to a BEAMing PCR method.

In a still further aspect of the invention, the microvesicle fraction of any of the foregoing methods is enriched for microvesicles originating from a specific cell type, such as, but not limited to brain, skin, or blood cells. In certain of the foregoing methods, a microvesicular surface molecule (e.g., but not limited to a surface antigen associated with tumor cells) is used to enrich for microvesicles from a specific cell type. In certain of these methods, the microvesicular surface molecule is epithelial-cell-adhesion-molecule (EpCAM), CD24, CD70, carcinoembryonic antigen (CEA), EGFR, EGFRvIII and other variants, Fas ligand, TRAIL, transferrin receptor, p38.5, p97, or HSP72. In a variation of the foregoing methods, the absence of a microvesicular surface molecule (such as, but not limited to CD80 or CD86) is used to enrich for microvesicles from a specific cell type. In the foregoing methods, the isolation of microvesicles from a specific cell type is accomplished by using antibodies, aptamers, aptamer analogs, or molecularly imprinted polymers.

In another aspect of the invention, any of the foregoing methods may include a microvesicle fraction obtained by one or more centrifugation procedures. In any of these methods, the one or more centrifugation procedures are performed at a speed not exceeding about 200,000 g; at a speed of about 2,000 g to about 200,000 g; at a speed not exceeding about 50,000 g; or at a speed not exceeding about 20,000 g.

Another aspect of the invention is a method for aiding the assessment of the hybridization efficiency of an oligo and its target sequence including the steps of (a) providing a first target sequence-specific primer labeled with a first tag; (b) providing a second target sequence-specific primer; (c) generating with the first and the second primers target sequence amplicons labeled with the first tag; (d) providing a medium coated with a second tag with affinity to the first tag; (e) mixing the amplicons labeled with the first tag with the medium coated with the second tag, thereby obtaining a medium coated with amplicons for assessing the hybridization efficiency of an oligo and its target sequence. In some aspect of the foregoing method; in other aspects of the foregoing method, the first tag is avidin (e.g., but not limited to streptavidin) and the second tag is biotin. In some aspects of the foregoing methods, the medium coated with a second tag is a bead (e.g., but not limited to a glass bead). In some aspects of the foregoing methods, the oligo is a probe for BEAMing PCR analysis (e.g., but not limited to, a fluorescently labeled probe).

### BRIEF DESCRIPTION OF THE DRAWINGS

FIG. 1 depicts the amplicon of the IDH1 gene that was used for BEAMing PCR analysis (SEQ ID NO: 1). The bold and italicized nucleotides represent the binding site for fluorescent probes specific for WT or G395A mutant sequence. The bold and non-italicized nucleotides represent the binding site for control fluorescent probe. The underlined nucleotide indicates the position of the G395A mutation.

FIG. 2 depicts a flow chart for a modified oligohybridization step in BEAMing PCR.

FIG. 3 is a representative FACS plot of a BEAMing PCR result from an assay designed to detect the G395A mutation within the IDH1 gene using microvesicles from a healthy individual. The X axis refers to the WT sequence signals. The Y axis refers to the G395A mutant sequence signals.

FIG. 4 is a representative FACS plot similar to the plot shown in FIG. 3 except that the microvesicles were from a glioma patient with wild-type IDH1 gene.

FIG. 5 is a representative FACS plot similar to the plot shown in FIG. 3 except that the microvesicles were from a glioma patient with G395A mutant IDH1 gene.

### DETAILED DESCRIPTION OF THE INVENTION

Microvesicles are shed by eukaryotic cells, or budded off of the plasma membrane, to the exterior of the cell. These membrane vesicles are heterogeneous in size with diameters ranging from about 10 nm to about 5000 nm. The small microvesicles (approximately 10 to 1000 nm, and more often approximately 30 to 200 nm in diameter) that are released by exocytosis of intracellular multivesicular bodies are referred to in the art as “exosomes.” Microvesicles can also be formed as apoptotic bodies during programmed cell death (Halicka et al., 2000). In addition, defective (i.e., non-infectious without helper-virus) retrovirus particles derived from human endogenous retroviral (HERV) elements may be found within microvesicle populations (Voisset et al., 2008). Exosomes, shedding microvesicles, microparticles, nanovesicles, apoptotic bodies, nanoparticles and membrane vesicles co-isolate using various techniques and will, therefore, collectively be referred to throughout this specification as “microvesicles” unless otherwise expressly denoted. The methods and compositions described herein are equally applicable to microvesicles of all sizes; preferably 30 to 800 nm; and more preferably 30 to 200 nm.

In some of the literature, the term “exosome” also refers to protein complexes containing exoribonucleases which are involved in mRNA degradation and the processing of small nucleolar RNAs (snoRNAs), small nuclear RNAs (snRNAs) and ribosomal RNAs (rRNA) (Liu et al., 2006; van Dijk et al., 2007). Such protein complexes do not have membranes and are not “microvesicles” or “exosomes” as those terms are used herein.

Certain aspects of the present invention are based on the finding that the nucleic acids found within microvesicles can be used as valuable biomarkers for tumor diagnosis, characterization and prognosis by providing a genetic biomarker or profile. The nucleic acids within microvesicles can also be used to monitor tumor progression over time by analyzing if other mutations are acquired during tumor progression as well as if the levels of certain mutations are increasing or decreasing over time or over a course of treatment. See (Skog et al., 2008) and WO 2009/100029.

Certain aspects of the present invention are based on the finding that the ability to analyze nucleic acids from microvesicles provides a non-invasive and sensitive method for detecting genetic aberrations. This ability to detect genetic aberrations provides for the ability to detect, diagnose, monitor, treat, or evaluate a disease or other medical condition, by analyzing nucleic acid content from microvesicles. Moreover, nucleic acids from microvesicles may be isolated and analyzed periodically as a means to detect changes in the nucleic acids. Such analyses can provide valuable information regarding the state of a disease or other medical condition, at the particular point in time that the microvesicles were obtained from the subject. This information may be used to assist in the therapeutic evaluation and decision-making process for a subject having a disease or other medical condition. For example, the presence or absence of one or more mutations in a particular gene may indicate the susceptibility to, presence of, or progression of a disease or other medical condition in a subject, or may indicate the likelihood that a particular therapeutic treatment will be efficacious.

Certain aspects of the present invention are based on another finding that most of the extracellular RNAs in bodily fluid from a subject are contained within microvesicles and thus protected from degradation by ribonucleases. More than 90% of extracellular RNA in total serum can be recovered in microvesicles. See (Skog et al., 2008) and WO 2009/100029.

In general terms, the present invention relates to methods for diagnosing, prognosing, monitoring, and treating a disease or other medical condition in a subject comprising the steps of, isolating, obtaining or using a microvesicle fraction from a bodily fluid of a subject, and analyzing one or more nucleic acids contained within the microvesicle fraction. The nucleic acids are analyzed qualitatively and/or quantitatively, and the results are compared to results expected or obtained for one or more other subjects who have or do not have the disease or other medical condition, or from the same subject at an earlier point in time. The presence of a difference in microvesicular nucleic acid content of the subject, as compared to a reference (e.g., microvesicular nucleic acid content of one or more other individuals, or prior analyses of the microvesicular nucleic content of the same individual) can indicate the presence or absence of a disease or other medical condition, the progression of said disease or other medical condition (e.g., changes of tumor size and tumor malignancy), the susceptibility to a disease or other medical condition, or the efficacy of a drug or other therapeutic treatment for a particular subject.

The step of isolating, obtaining or using a microvesicle fraction from a bodily fluid of a subject encompasses (1) the use of separation and/or enrichment techniques to isolate a microvesicle fraction from a bodily fluid such as serum, plasma or urine, as described in detail at various points below; (2) the simple act of obtaining a microvesicle fraction or preparation made by another from a bodily fluid of a subject; and (3) a combination of (1) and (2), e.g., wherein one obtains a microvesicle fraction or preparation from another and further refines it, e.g., by enriching for microvesicles of a certain type (e.g., by surface marker selection according to techniques described below).

The compositions, methods and techniques described herein provide the following advantages: 1) the opportunity to selectively analyze disease- or tumor-specific nucleic acids, which may be realized by isolating disease- or tumor-specific microvesicles apart from other microvesicles within the fluid sample; 2) significantly higher yield of nucleic acid species with higher sequence integrity as compared to the yield/integrity obtained by extracting nucleic acids directly from the fluid sample; 3) scalability, e.g. to detect nucleic acids expressed at low levels, the sensitivity can be increased by isolating more microvesicles from a larger volume of serum; 4) purer nucleic acids in that protein and lipids, debris from dead cells, and other potential contaminants and PCR inhibitors are excluded from the microvesicle preparation before the nucleic acid extraction step; and 5) more choices in nucleic acid extraction methods as microvesicle preparations are of much smaller volume than that of the starting serum, making it possible to extract nucleic acids from these microvesicle preparations using small volume column filters.

The microvesicles are preferably isolated from a bodily fluid from a subject. As used herein, a “bodily fluid” refers to a sample of fluid isolated from anywhere in the body of the subject, preferably a peripheral location, including but not limited to, blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof.

The term “biological sample” includes, for example, a cell, a group of cells, fragments of cells, cell products including for example microvesicles, cell cultures, bodily tissues from a subject, or bodily fluids (as defined above).

The term “subject” is intended to include all animals shown to or expected to have microvesicles. In particular embodiments, the subject is a mammal, a human or nonhuman primate, a dog, a cat, a horse, a cow, other farm animals, or a rodent (e.g. mice, rats, guinea pig, etc.). The term “subject” and “individual” are used interchangeably herein.

Methods of isolating microvesicles from a biological sample are known in the art. For example, a method of differential centrifugation is described in a paper by Raposo, et al. (Raposo et al., 1996), and similar methods are detailed in the Examples section herein. Methods of anion exchange and/or gel permeation chromatography are described in U.S. Pat. Nos. 6,899,863 and 6,812,023. Methods of sucrose density gradients or organelle electrophoresis are described in U.S. Pat. No. 7,198,923. A method of magnetic activated cell sorting (MACS) is described in (Taylor and Gercel-Taylor, 2008). A method of nanomembrane ultrafiltration concentrator is described in (Cheruvanky et al., 2007). Microvesicles can be identified and isolated from the bodily fluid of a subject by microchip technology, as described for example in (Nagrath et al., 2007).

Further, methods for isolating microvesicles from a biological sample and extracting biological materials from the isolated microvesicles are also described in this application as well as in scientific publications and patent applications, e.g. (Chen et al., 2010; Miranda et al., 2010; Skog et al., 2008). See also WO 2009/100029, WO 2011/009104, WO 2011/031892 and WO 2011/031877. These publications are incorporated herein by reference for their disclosures pertaining to isolation and extraction methods and techniques. Each of the foregoing references is incorporated by reference herein for its teaching of these methods.

In one embodiment, the microvesicles isolated from a bodily fluid are enriched for those originating from a specific cell type, for example, skin, brain, and blood cells. Because the microvesicles often carry surface molecules such as antigens from their donor cells, surface molecules may be used to identify, isolate and/or enrich for microvesicles from a specific donor cell type (Al-Nedawi et al., 2008; Taylor and Gercel-Taylor, 2008). In this way, microvesicles originating from distinct cell populations can be analyzed for their nucleic acid content. For example, tumor (malignant and non-malignant) microvesicles carry tumor-associated surface antigens and may be detected, isolated and/or enriched via these specific tumor-associated surface antigens. In one example, the surface antigen is epithelial-cell-adhesion-molecule (EpCAM), which is specific to microvesicles from carcinomas of lung, colorectal, breast, prostate, head and neck, and hepatic origin, but not of hematological cell origin (Balzar, et al. 1999; Went, et al. 2004). In another example, the surface antigen is CD24, which is a glycoprotein specific to urine microvesicles (Keller, et al. 2007). In yet another example, the surface antigen is selected from a group of molecules CD70, carcinoembryonic antigen (CEA), EGFR, EGFRvIII and other variants, Fas ligand, TRAIL, tranferrin receptor, p38.5, p97 and HSP72. Additionally, tumor specific microvesicles may be characterized by the lack of surface markers, such as CD80 and CD86.

The isolation of microvesicles from specific cell types can be accomplished, for example, by using antibodies, aptamers, aptamer analogs or molecularly imprinted polymers specific for a desired surface antigen. In one embodiment, the surface antigen is specific for a cancer type. In another embodiment, the surface antigen is specific for a cell type which is not necessarily cancerous. One example of a method of microvesicle separation based on cell surface antigen is provided in U.S. Pat. No. 7,198,923. As described in, e.g., U.S. Pat. Nos. 5,840,867 and 5,582,981, WO 2003/050290 and a publication by Johnson, et al. (Johnson et al., 2008), aptamers and their analogs specifically bind surface molecules and can be used as a separation tool for retrieving cell type-specific microvesicles. Molecularly imprinted polymers also specifically recognize surface molecules as described in, e.g., U.S. Pat. Nos. 6,525,154, 7,332,553 and 7,384,589 and a publication by Bossi, et al. (Bossi et al., 2007) and are a tool for retrieving and isolating cell type-specific microvesicles. Each of the foregoing reference is incorporated herein for its teaching of these methods.

It may be beneficial or otherwise desirable to extract the nucleic acid from the exosomes prior to the analysis. Nucleic acid molecules can be isolated from a microvesicle using any number of procedures, which are well-known in the art, the particular extraction procedure chosen being appropriate for the particular biological sample. For example, methods for extracting high quality nucleic acids microvesicles are described in our prior patent applications U.S. 61/412,369 filed on Nov. 10, 2010 and U.S. 61/485,112 filed on May 11, 2011, each of which is incorporated herein for its teaching of these methods. In some instances, with some techniques, it may also be possible to analyze the nucleic acid without extraction from the microvesicle.

In one embodiment, the extracted nucleic acids, including DNA and/or RNA, are analyzed directly without an amplification step. Direct analysis may be performed with different methods including, but not limited to, nanostring technology. NanoString technology enables identification and quantification of individual target molecules in a biological sample by attaching a color coded fluorescent reporter to each target molecule. This approach is similar to the concept of measuring inventory by scanning barcodes. Reporters can be made with hundreds or even thousands of different codes allowing for highly multiplexed analysis. The technology is described in a publication by Geiss, et al. (Geiss et al., 2008) and is incorporated herein by reference for this teaching.

In another embodiment, it may be beneficial or otherwise desirable to amplify the nucleic acid of the microvesicle prior to analyzing it. Methods of nucleic acid amplification are commonly used and generally known in the art, many examples of which are described herein. If desired, the amplification can be performed such that it is quantitative. Quantitative amplification will allow quantitative determination of relative amounts of the various nucleic acids, to generate a profile as described below.

In one embodiment, the extracted nucleic acid is DNA. In another embodiment, the extracted nucleic acid is RNA. RNAs are preferably reverse-transcribed into complementary DNAs (“cDNA”). Such reverse transcription may be performed alone or in combination with an amplification step. One example of a method combining reverse transcription and amplification steps is reverse transcription polymerase chain reaction (RT-PCR), which may be further modified to be quantitative, e.g., quantitative RT-PCR as described in U.S. Pat. No. 5,639,606, which is incorporated herein by reference for this teaching.

Nucleic acid amplification methods include, without limitation, polymerase chain reaction (PCR) (U.S. Pat. No. 5,219,727) and its variants such as in situ polymerase chain reaction (U.S. Pat. No. 5,538,871), quantitative polymerase chain reaction (U.S. Pat. No. 5,219,727), nested polymerase chain reaction (U.S. Pat. No. 5,556,773), self sustained sequence replication and its variants (Guatelli et al., 1990), transcriptional amplification system and its variants (Kwoh et al., 1989), Qb Replicase and its variants (Miele et al., 1983), cold-PCR (Li et al., 2008) or any other nucleic acid amplification methods, followed by the detection of the amplified molecules using techniques known to those of skill in the art. Especially useful are those detection schemes designed for the detection of nucleic acid molecules if such molecules are present in very low numbers. The foregoing references are incorporated herein for their teachings of these methods.

The analysis of nucleic acids present in the microvesicles is quantitative, qualitative, or both quantitative and qualitative. For quantitative analysis, the amounts (expression levels), either relative or absolute, of specific nucleic acids of interest within the microvesicles are measured with methods known in the art. For qualitative analysis, the species of specific nucleic acids of interest within the microvesicles, whether wild type or variants, are identified with methods known in the art.

“Genetic aberrations” is used herein to refer to the nucleic acid amounts as well as nucleic acid variants within the microvesicles. Specifically, genetic aberrations include, without limitation, over-expression of a gene (e.g., oncogenes), under-expression of a gene (e.g., tumor suppressor genes), alternative production of splice variants of a gene or a panel of genes, gene copy number variants (CNV) (e.g. DNA double minutes) (Hahn, 1993), nucleic acid modifications (e.g., methylation, acetylation and phosphorylations), single nucleotide polymorphisms (SNPs), chromosomal rearrangements (e.g., inversions, deletions and duplications), and mutations (insertions, deletions, duplications, missense, nonsense, synonymous or any other nucleotide changes) of a gene or a panel of genes, which mutations, in many cases, ultimately affect the activity and function of the gene products, lead to alternative transcriptional splicing variants and/or changes of gene expression level.

Aspects of the invention relate to the detection by the methods described herein, of the presence or absence of one or more nucleotide variants of a gene specific to a disease (e.g., a cancer), or for an increase or decrease in nucleic acid levels specific to a disease (e.g., a cancer). Such nucleotide variants and differences in nucleic acid levels are typically referred to in the art as disease associated genetic aberrations, a variety of which are or referred to herein. The detection of the presence of the nucleotide variant or an increase or decrease in nucleic acid level, indicates the presence of the disease (e.g., the cancer) in the individual.

The determination of such genetic aberrations can be performed by a variety of techniques known to the skilled practitioner. For example, expression levels of nucleic acids, alternative splicing variants, chromosome rearrangement and gene copy numbers can be determined by microarray analysis (U.S. Pat. Nos. 6,913,879, 7,364,848, 7,378,245, 6,893,837 and 6,004,755) and quantitative PCR. Particularly, copy number changes may be detected with the Illumina Infinium II whole genome genotyping assay or Agilent Human Genome CGH Microarray (Steemers et al., 2006). Nucleic acid modifications can be assayed by methods described in, e.g., U.S. Pat. No. 7,186,512 and patent publication WO 2003/023065. Particularly, methylation profiles may be determined by Illumina DNA Methylation OMA003 Cancer Panel. SNPs and mutations can be detected by hybridization with allele-specific probes, enzymatic mutation detection, chemical cleavage of mismatched heteroduplex (Cotton et al., 1988), ribonuclease cleavage of mismatched bases (Myers et al., 1985), mass spectrometry (U.S. Pat. Nos. 6,994,960, 7,074,563, and 7,198,893), nucleic acid sequencing, single strand conformation polymorphism (SSCP) (Orita et al., 1989), denaturing gradient gel electrophoresis (DGGE) (Fischer and Lerman, 1979a; Fischer and Lerman, 1979b), temperature gradient gel electrophoresis (TGGE) (Fischer and Lerman, 1979a; Fischer and Lerman, 1979b), restriction fragment length polymorphisms (RFLP) (Kan and Dozy, 1978a; Kan and Dozy, 1978b), oligonucleotide ligation assay (OLA), allele-specific PCR (ASPCR) (U.S. Pat. No. 5,639,611), ligation chain reaction (LCR) and its variants (Abravaya et al., 1995; Landegren et al., 1988; Nakazawa et al., 1994), flow-cytometric heteroduplex analysis (WO/2006/113590) and combinations or modifications of any of the foregoing.

For a further example, digital PCR is used to determine genetic aberrations. A digital PCR technique amplifies a single DNA template from minimally diluted DNA samples, thereby generating amplicons that are exclusively derived from one template and can be detected with different fluorophores or sequencing to discriminate different alleles. Thus, digital PCR transforms the exponential, analog signals obtained from conventional PCR to linear, digital signals, allowing statistical analysis of the PCR product. Digital PCR has been applied in quantification of mutant alleles and detection of allelic imbalance in clinical specimens, providing a promising molecular diagnostic tool for cancer detection. See, e.g., a review article entitled “Principle and applications of digital PCR” by Pohl and Shih, 2004.

One type of digital PCR is based on the technique known as BEAMing (beads, emulsion, amplification, and magnetics) and may be used to detect rare genetic aberrations (Diehl et al., 2006). In BEAMing PCR, water-in-oil droplets containing primer-coated beads, templates, and reaction components are generated such that each droplet ideally contains one template. This allows for the amplification of solely a mutant or wild-type template onto the respective bead. After the PCR is completed, the droplets are broken and the beads are purified so that the identity of the attached DNA can be interrogated with fluorescent probes or otherwise labeled probes. Probe-bound beads are then analyzed by flow cytometry and counted as wild-type or mutant events. In this way, BEAMing PCR allows for the conversion of the normally exponential PCR signal into a digital one, thereby enabling high sensitivity and quantification of the percentage of a starting population that is mutant.

In general, the methods for analyzing genetic aberrations are reported in numerous publications, not limited to those cited herein, and are available to skilled practitioners. The appropriate method of analysis will depend upon the specific goals of the analysis, the condition/history of the patient, and the specific cancer(s), diseases or other medical conditions to be detected, monitored or treated. The forgoing references are incorporated herein for their teachings of these methods.

The published literature describes a variety of genetic aberrations that have been identified to occur and/or contribute to the initial generation or progression of cancer. Examples of genes which are commonly under expressed, or over expressed in brain tumors are reviewed in (Furnari et al., 2007), and this subject matter is incorporated herein by reference. Therefore, in some embodiments, the presence or absence of an increase or decrease in the nucleic acid expression level of a gene(s) and/or a microRNA(s) whose disregulated expression level is specific to a type of cancer can be used to indicate the presence or absence of the type of cancer in the subject.

Likewise, nucleic acid variants, e.g., DNA or RNA modifications, single nucleotide polymorphisms (SNPs) and mutations (e.g., missense, nonsense, insertions, deletions, duplications) may also be analyzed within microvesicles from bodily fluid of a subject, including pregnant females where microvesicles derived from the fetus may be in serum as well as amniotic fluid.

In addition, more genetic aberrations associated with cancers have been identified recently in some research projects. For example, the Cancer Genome Atlas (TCGA) program explores a spectrum of genomic changes involved in human cancers. The results of this project and other similar research efforts are published and incorporated herein by reference (Jones et al., 2008; McLendon et al., 2008; Parsons et al., 2008; Wood et al., 2007). Specifically, these research projects have identified genetic aberrations, such as mutations (e.g., missense, nonsense, insertions, deletions and duplications), gene expression level variations (mRNA or microRNA), copy number variations and nucleic acid modification (e.g. methylation), in human glioblastoma, pancreatic cancer, breast cancer and/or colorectal cancer. Any genetic aberrations associated with cancer are targets that may be selected for use in diagnosing and/or monitoring cancer by the methods described herein.

Detection of one or more nucleotide variants can be accomplished by performing a nucleotide variant screen on the nucleic acids within the microvesicles. Such a screen can be as wide or narrow as determined necessary or desirable by the skilled practitioner. It can be a wide screen (set up to detect all possible nucleotide variants in genes known to be associated with one or more cancers or disease states). Where one specific cancer or disease is suspected or known to exist, the screen can be specific to that cancer or disease. One example is a brain tumor/brain cancer screen (e.g., set up to detect all possible nucleotide variants in genes associated with various clinically distinct subtypes of brain cancer or known drug-resistant or drug-sensitive mutations of that cancer).

Which nucleic acids are to be amplified and/or analyzed can be selected by the skilled practitioner. The entire nucleic acid content of the exosomes or only a subset of specific nucleic acids which are likely or suspected of being influenced by the presence of a disease or other medical condition such as cancer, can be amplified and/or analyzed. The identification of a genetic aberration(s) in the analyzed microvesicle nucleic acid can be used to diagnose the subject for the presence of a disease such as cancer, hereditary diseases or viral infection with which that aberration(s) is associated.

In one embodiment, mutations of a gene which is associated with a disease such as cancer (e.g., via nucleotide variants, over-expression or under-expression) are detected by analysis of nucleic acids in microvesicles. The nucleic acid sequences may be complete or partial, as both are expected to yield useful information in diagnosis and prognosis of a disease. The sequences may be sense or anti-sense to the actual gene or transcribed sequences. The skilled practitioner will be able to devise detection methods for a nucleotide variance from either the sense or anti-sense nucleic acids which may be present in a microvesicle. Many such methods involve the use of probes which are specific for the nucleotide sequences which directly flank, or contain the nucleotide variances. Such probes can be designed by the skilled practitioner given the knowledge of the gene sequences and the location of the nucleic acid variants within the gene. Such probes can be used to isolate, amplify, and/or actually hybridize to detect the nucleic acid variants, as described in the art and herein.

In further embodiments of the present invention, microvesicle fractions were analyzed to detect genetic aberrations in one or more genes or the pathways in which the genes are involved, including but not limited to, IDH1, IDH2, TP53, PTEN, CDKN2A, NF1, EGFR, RB1, PIK3CA, BRAF, and the pathways in which each of the above genes is involved. Genetic aberrations in these genes have been found in cells, tissues, or organs with diseases or other medical conditions, e.g., glioma. For example, somatic mutations of codon 132 of the isocitrate dehydrogenase 1 gene (IDH1) were found in about 83% of secondary glioblastoma samples. Codon 132 mutation may cause various amino acid changes in IDH1 protein, e.g., R132H, R132C, R132S, R132L, and R132G. See WO 2010/028099. Therefore, in one embodiment of the present invention, the W395A mutation in the IDH1 gene that leads to the R132H change in the IDH1 protein is detected in the nucleic acids extracted from a microvesicle fraction isolated from a patient blood using a BEAMing PCR technique. This detection method is described in detail in the Example section of the present application.

Determining the presence or absence of a particular nucleotide variant or plurality of variants in the nucleic acid within microvesicles from a subject can be performed in a variety of ways. A variety of methods are available for such analysis, including, but not limited to, PCR, hybridization with allele-specific probes, enzymatic mutation detection, chemical cleavage of mismatches, mass spectrometry or DNA sequencing, including minisequencing. In particular embodiments, hybridization with allele specific probes can be conducted in two formats: 1) allele specific oligonucleotides bound to a solid phase (glass, silicon, nylon membranes) and the labeled sample in solution, as in many DNA chip applications, or 2) bound sample (often cloned DNA or PCR amplified DNA) and labeled oligonucleotides in solution (either allele specific or short so as to allow sequencing by hybridization). Diagnostic tests may involve a panel of variances, often on a solid support, which enables the simultaneous determination of more than one variance. In another embodiment, determining the presence of at least one nucleic acid variance in the microvesicle nucleic acid entails a haplotyping test. Methods of determining haplotypes are known to those of skill in the art, as for example, in WO 00/04194.

In one embodiment, the determination of the presence or absence of a nucleic acid variant(s) involves determining the sequence of the variant site or sites (the exact location within the sequence where the nucleic acid variation from the norm occurs) by methods such as polymerase chain reaction (PCR), chain terminating DNA sequencing (U.S. Pat. No. 5,547,859), minisequencing (Fiorentino et al., 2003), oligonucleotide hybridization, pyrosequencing, Illumina genome analyzer, deep sequencing, mass spectrometry or other nucleic acid sequence detection methods. Methods for detecting nucleic acid variants are well known in the art and some of the methods are disclosed in WO 00/04194, incorporated herein by reference. In an exemplary method, the diagnostic test comprises amplifying a segment of DNA or RNA (generally after converting the RNA to complementary DNA) spanning one or more known variants in the desired gene sequence. This amplified segment is then sequenced and/or subjected to electrophoresis in order to identify nucleotide variants in the amplified segment.

In one embodiment, the invention provides a method of screening for nucleotide variants in the nucleic acid of microvesicles isolated as described herein. This can be achieved, for example, by PCR or, alternatively, in a ligation chain reaction (LCR) (Abravaya et al., 1995; Landegren et al., 1988; Nakazawa et al., 1994). LCR can be particularly useful for detecting point mutations in a gene of interest (Abravaya et al., 1995). The LCR method comprises the steps of designing degenerate primers for amplifying the target sequence, the primers corresponding to one or more conserved regions of the nucleic acid corresponding to the gene of interest, amplifying PCR products with the primers using, as a template, a nucleic acid obtained from a microvesicle, and analyzing the PCR products. Comparison of the PCR products of the microvesicle nucleic acid to a control sample (either having the nucleotide variant or not) indicates variants in the microvesicle nucleic acid. The change can be either an absence or presence of a nucleotide variant in the microvesicle nucleic acid, depending upon the control.

Many methods of diagnosis performed on a tumor biopsy sample can be performed with microvesicles since tumor cells are known to shed microvesicles into bodily fluid and the genetic aberrations within these microvesicles reflect those within tumor cells as demonstrated herein. Furthermore, methods of diagnosis using microvesicles have characteristics that are absent in methods of diagnosis performed directly on a tumor biopsy sample. For example, one particular advantage of the analysis of microvesicular nucleic acids, as opposed to other forms of sampling of tumor/cancer nucleic acid, is the availability for analysis of tumor/cancer nucleic acids derived from all foci of a tumor or genetically heterogeneous tumors present in an individual. Biopsy samples are limited in that they provide information only about the specific focus of the tumor from which the biopsy is obtained. Different tumorous/cancerous foci found within the body, or even within a single tumor often have different genetic profiles and are not analyzed in a standard biopsy. However, analysis of the microvesicular nucleic acids from an individual presumably provides a sampling of all foci within an individual. This provides valuable information with respect to recommended treatments, treatment effectiveness, disease prognosis, and analysis of disease recurrence, which cannot be provided by a simple biopsy.

In one embodiment, the microvesicle fraction from a bodily fluid of a subject is pre-treated with DNase, RNase inhibitor, or a combination of DNase and RNase inhibitor to eliminate or substantially eliminate all of materials that adversely affect the quantity, quality, or both quantity and quality of nucleic acid extractions. The pre-treatment is sometimes preferred when a high quality of nucleic acid extraction from microvesicles is desired. For example, when a bodily fluid sample does not generally give rise to good nucleic acid extraction from microvesicles for the sample, a pre-treatment step such as described above may be used to improve the quantity, quality, or both quantity and quality of the extraction.

Identification of genetic aberrations associated with specific diseases and/or medical conditions by the methods described herein can also be used for prognosis, monitoring, or aiding in the making of treatment decisions for an individual diagnosed with a disease or other medical condition such as cancer. For example, mutations in the isocitrate dehydrogenase gene (IDH1) were recently described in patients with acute myeloid leukemia (AML) and an IDH1 mutation was an independent adverse prognostic factor for relapse in FLT3/ITD(−) patients and a favorable factor in FLT3/ITD(+) patients (Green et al., 2010). In addition, IDH1 status was found to be more prognostic for overall survival than standard histological criteria that differentiate high-grade astrocytomas (Hartmann et al., 2010). Such nucleotide variants can be identified in nucleic acids present in microvesicles by the methods described herein.

In some embodiments where the invention is used for purpose of monitoring the progression of a disease or other medical condition such as a cancer, the process of detecting an associated genetic aberration is performed periodically over time, and the results reviewed, to monitor the progression or regression of the disease. Put another way, a change in the genetic aberration indicates a change in the disease state in the subject. The period of time to elapse between sampling of microvesicles from the subject, for performance of the isolation and analysis of the microvesicle, will depend upon the circumstances of the subject, and is to be determined by the skilled practitioner.

Selection of an individual from whom the microvesicles are isolated is performed by the skilled practitioner based upon analysis of one or more of a variety of factors. Such factors for consideration are whether the subject has a family history of a specific disease (e.g. a cancer), has a genetic predisposition for such a disease, has an increased risk for such a disease due to family history, genetic predisposition, other disease or physical symptoms which indicate a predisposition, or environmental reasons. Environmental reasons include lifestyle, exposure to agents which cause or contribute to the disease such as in the air, land, water or diet. In addition, having previously had the disease, being currently diagnosed with the disease prior to therapy or after therapy, being currently treated for the disease (undergoing therapy), being in remission or recovery from the disease, are other reasons to select an individual for performing the methods.

All patents, patent applications, and publications identified herein are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs:
1. A method for assaying a biological sample, comprising the steps of:
   (a) isolating, obtaining or using a microvesicle fraction from a biological sample; and
   (b) detecting in the microvesicle fraction the presence or absence of a genetic aberration in a gene selected from the group consisting of IDH1, IDH2, TP53, PTEN, CDKN2A, NF1, EGFR, RB1, PIK3CA, and BRAF.
2. A method for aiding in diagnosis, prognosis, monitoring, or therapy selection in relation to a disease or other medical condition in a subject, comprising the steps of:
   (a) isolating, obtaining or using a microvesicle fraction from a bodily fluid from a subject; and
   (b) detecting in the microvesicle fraction the presence or absence of a genetic aberration in a gene selected from the group consisting of IDH1, IDH2, TP53, PTEN, CDKN2A, NF1, EGFR, RB1, PIK3CA, and BRAF, wherein the genetic aberration is associated with the diagnosis, prognosis, monitoring, or therapy selection in relation to a disease or other medical condition.
3. The method of any of paragraphs 1-2, further comprising a step of extracting nucleic acids from the microvesicle fraction prior to detection of the genetic aberration.
4. The method of paragraph 3, further comprising a step of treating the microvesicle fraction with DNase, RNAse inhibitor, or a combination of DNase and RNase inhibitor prior to or together with the step of extracting nucleic acids from the microvesicle fraction.
5. The method of paragraph 3 or paragraph 4, wherein the extracted nucleic acid is RNA.
6. The method of paragraph 5, wherein the RNA is reverse-transcribed into complementary DNA.
7. The method of any of paragraphs 3-6, wherein the nucleic acid is amplified prior to analysis.
8. The method of paragraph 7, wherein the nucleic acid amplification is carried out by polymerase chain reaction (PCR) or any of its variants such as in situ PCR, quantitative PCR, nested PCR; self-sustained sequence replication or any of its variants; transcriptional amplification system or any of its variants; Qb Replicase or any of its variants; or cold-PCR.
9. The method of paragraph 1 or paragraph 2, wherein the detection of the presence or absence of a genetic aberration is performed using a digital PCR method.
10. The method of paragraph 9, wherein the digital PCR method is a BEAMing PCR method.
11. The method of any of paragraphs 1-10, wherein the gene is IDH1
12. The method of paragraph 11, wherein the genetic aberration is the G295A mutation.
13. The method of any of paragraphs 1-12, wherein the disease or other medical condition is cancer.
14. The method of paragraph 13, wherein the cancer is glioma, leukemia, or melanoma.
15. The method of paragraph 14, wherein the glioma is astrocytomas, oligodendrogliomas, oligoastrocytomas, or secondary glioblastomas.
16. The method of any of paragraphs 1-15, wherein the bodily fluid is blood, plasma, serum, urine, or a combination thereof.
17. The method of any of paragraphs 1-16, wherein the subject is a human.
18. The method of any of paragraphs 1-17, wherein the microvesicle fraction is enriched for microvesicles originating from a specific cell type.
19. The method of paragraph 18, wherein the specific cell type is brain, skin, or blood cells.
20. The method of paragraph 18 or paragraph 19, wherein a microvesicular surface molecule is used to enrich for microvesicles from a specific cell type.
21. The method of paragraph 20, wherein the microvesicular surface molecule is a surface antigen associated with tumor cells.
22. The method of paragraph 21, wherein the microvesicular surface molecule is epithelial-cell-adhesion-molecule (EpCAM), CD24, CD70, carcinoembryonic antigen (CEA), EGFR, EGFRvIII and other variants, Fas ligand, TRAIL, transferrin receptor, p38.5, p97, or HSP72.
23. The method of paragraph 18, wherein the absence of a microvesicular surface molecule is used to enrich for microvesicles from a specific cell type.
24. The method of paragraph 23, wherein the absent surface molecule is CD80 or CD86.
25. The method of any of paragraphs 18-24, wherein the isolation of microvesicles from a specific cell type is accomplished by using antibodies, aptamers, aptamer analogs, or molecularly imprinted polymers.
26. The method of any of paragraphs 1-15, wherein the microvesicle fraction is obtained by one or more centrifugation procedures.
27. The method of paragraph 26, wherein the one or more centrifugation procedures are performed at a speed not exceeding about 200,000 g.
28. The method of paragraph 27, wherein the one or more centrifugation procedures are performed at a speed of about 2,000 g to about 200,000 g.
29. The method of paragraph 28, wherein the one or more centrifugation procedures are performed at a speed not exceeding about 50,000 g.
30. The method of paragraph 28, wherein the centrifugation procedures are performed at a speed not exceeding about 20,000 g.
31. A method for aiding the assessment of the hybridization efficiency of an oligo and its target sequence, comprising the steps of:
   a. providing a first target sequence-specific primer labeled with a first tag;
   b. providing a second target sequence-specific primer;
   c. generating with the first and the second primers target sequence amplicons labeled with the first tag;
   d. providing a medium coated with a second tag with affinity to the first tag;
   e. mixing the amplicons labeled with the first tag with the medium coated with the second tag, thereby obtaining a medium coated with amplicons for assessing the hybridization efficiency of an oligo and its target sequence.
32. The method of paragraph 31, wherein the first tag is a biotin and the second tag is avidin.
33. The method of paragraph 31, wherein the first tag is avidin and the second tag is biotin.
34. The method of any of paragraphs 32 and 33, wherein the avidin is streptavidin
35. The method of paragraph 31, wherein the medium coated with a second tag is a bead.
36. The method of paragraph 35, wherein the bead is a glass bead.
37. The method of paragraph 31, wherein the oligo is a probe for BEAMing PCR analysis.
38. The method of paragraph 37, wherein the probe is fluorescently labeled.

The invention is further illustrated by the following examples, which should not be construed as further limiting.

### Example

### Method of Assaying IDH1 G395A Mutation Using Microvesicles Isolated from Serum Samples

Serum samples were obtained according to standard procedures from healthy individuals, glioma patients with wild-type IDH1, and glioma patients with G395A mutant IDH1. Microvesicles were isolated from the serum samples and nucleic acids were then extracted from the isolated microvesicles as described below.

One milliliter of serum was transferred into a 1.5 ml Eppendorf tube containing 8 μl SuperaseIn RNase inhibitor (Ambion Inc.). After a 20,000 g, 0.5 hour centrifugation step, the pellet was used for nucleic acid extraction employing a modified miRNeasy RNA extraction protocol version 3.0.

In this modified protocol, we used a mixture of DNAse/SuperaseIn RNase inhibitor to treat the pellet (DNase was from the DNA Free Turbo kit; both DNase and SuperaseIn were from Ambion, Inc.). The DNase could be optionally replaced by an on-column DNase step following the miRNeasy protocol. This on-column treatment removes most of the DNA in the extraction, including DNA that is potentially inside the isolated microvesicles. These DNA may affect RNA integrity when the extracted RNA quantity is very small. If on-column DNase treatment is selected, the pellet is treated with 8 μL SuperaseIn in 42 μL PBS.

The mixture of DNase and SuperaseIn RNase inhibitor was made according to the following scheme:

| | |
|---|---|
| DNase I | 2 μL |
| DNase buffer (10X) | 5 μL |
| SuperaseIn | 8 μL |
| 1xPBS | 35 μL |
| | 50 μL |

DNase I and DNase buffer were from the DNA Free Turbo kit (Ambion). SuperaseIn RNase inhibitor (Ambion) was utilized at a concentration of 20 units/μL. The pellet was mixed with 50 μL of the DNase/SuperaseIn mixture as mentioned above and incubated at room temperature for 20 min in the centrifuge tube. Then 700 μl Qiazol lysis buffer (Qiagen) was added to each sample in the centrifuge tube and mixed by pipetting up and down 15 times to dissolve/re-suspend the pellet. The suspended pellet mixture was immediately transferred to an Eppendorf tube. Further nucleic acid extraction was then performed in a PCR hood. The tube with the pellet mixture was vortexed briefly and incubated at room temperature for 2-4 min before 140 μl chloroform was added into the tube containing the mixture. The tube was then capped, shaken vigorously for 20 seconds, incubated at room temperature for 2-3 min, and centrifuged for 15 min at 12,000 g at 4° C. The upper aqueous phase was transferred to a new collection tube into which, 1.5 volumes (usually 600 μl) of 100% ethanol was added and mixed thoroughly by pipetting up and down several times.

Up to 700 μl of the ethanol mixture, including any precipitate that may have formed, was transferred into an RNeasy Micro spin column (MinElute column stored @ +4° C., from the Qiagen RNeasy Micro kit). The spin column was inserted in a 2 ml collection tube as supplied by the manufacturer, and centrifuged at 1000 g for 15 seconds at room temperature. The flow-through was discarded. The centrifugation step was repeated until all the remaining mixture had been added. Again, the flow-through was discarded. The nucleic acids on the column were then washed three times as follows: 1) 700 μL Buffer RWT was added onto the RNeasy MinElute spin column and centrifuged for 15 seconds at 8500 g to wash the column (the flow-through was discarded); 2) 500 μL Buffer RPE was added onto the RNeasy MinElute spin column and centrifuged for 15 seconds at 8500 g to wash the column (the flow-through was discarded); 3) the Buffer RPE wash step was repeated except that the column was centrifuged for 2 minutes at 8500 g to dry the RNeasy Mini spin column membrane.

After the washing steps, the RNeasyMinElute spin column was inserted into a new 2 ml collection tube and centrifuged at 14000 g for 5 minutes to further dry the column membrane. The dried column was inserted into another new 1.5 ml collection tube and 16 μL RNase-free water was added onto the dried column membrane and incubated for 1 minute at room temperature. The ribonucleic acids (RNAs) were eluted by centrifugation for 1 minute at 8500 g. The volume of the eluted RNA was about 14 μl.

We reverse transcribed 12 μl of the extracted RNA into cDNA using Superscript VILO cDNA Synthesis Kit (Invitrogen 11754-050). The reverse transcription reaction mixture was made according to the following scheme (Table 1). The “5×” or “10×” indicates that the original concentration is 5 time or 10 times the final concentration in the reaction mixture, respectively. The unit “μl” is a shorthand for microliter.

TABLE 1
Reverse transcription reaction mixture scheme
for each reverse transcription reaction.

| Original reagent | Amount (μl) |
|---|---|
| 5X VILO ™ Reaction Mix | 4 |
| 10X Superscript ® Enzyme Mix | 2 |
| RNA (up to 2.5 μg) | 12 |
| Nuclease free water | 2 |
| Total volume | 20 |

The reverse transcription was performed in a Veriti PCR machine (Applied Biosystems) under the following conditions: 25° C. for 10 min, 42° C. for 70 min, 85° C. for 5 min, hold at 4° C. before storing the reaction at −20° C.

The reverse-transcribed cDNA was then processed with an initial round of standard PCR (pre-amplification step), and the resulting amplicons were used as input for further analysis using a modification of the conventional BEAMing PCR technique described in (Diehl et al., 2006).

The primers used for the pre-amplification step were: forward tcccgcgaaattaatacgacCGGTCTTCAGAGAAGCCATT (SEQ ID NO: 2) (lower case refers to TAG 1 sequence, upper case refers to the sequence that binds to IDH1 template); and gctggagctctgcagctaAGGCCCAGGAACAACAAAAT (SEQ ID NO: 3) (lower case refers to TAG 1 sequence, upper case refers to the sequence that binds to IDH1 template).

The primers used for the emulsion PCR were as follows. The TAG-1 sequence primer that is attached to streptavidin bead was 5′-dual biotin-PEGspacer18-ttcccgcgaaattaatacgac (SEQ ID NO: 4). The forward primer for emulsion PCR with 5′ modification was tcccgcgaaattaatacgac (SEQ ID NO: 5). The reverse primer for emulsion PCR was AATCAGTTGCTCTGTATTGATCC (SEQ ID NO: 6).

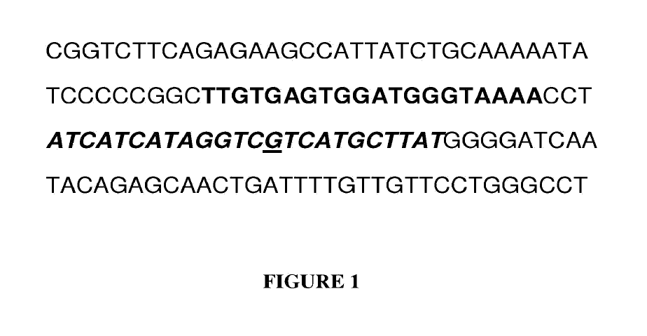

As shown in FIG. 1, the amplicon for IDH1 included the mutation site G395A. The binding site for fluorescent probes specific for WT or G395A sequence corresponded to ATCATCATAGGTCGTCATGCTTAT (SEQ ID NO: 7). The binding site for control fluorescent probe was TTGTGAGTGGATGGGTAAAA (SEQ ID NO: 8). Thus, the probe used for detecting wild-type sequence was ATAAGCATGACGACCTATGAT (SEQ ID NO: 9) which was labeled with fluorescent tag AF488. The probe used for detecting G395A mutant sequence was ATAAGCATGATGACCTATGAT (SEQ ID NO: 10) which was labeled with fluorescent tag AF647. The probe used for detecting G395A mutant sequence was TTTTACCCATCCACTCACAA (SEQ ID NO: 11) which was labeled with fluorescent tag pacific blue.

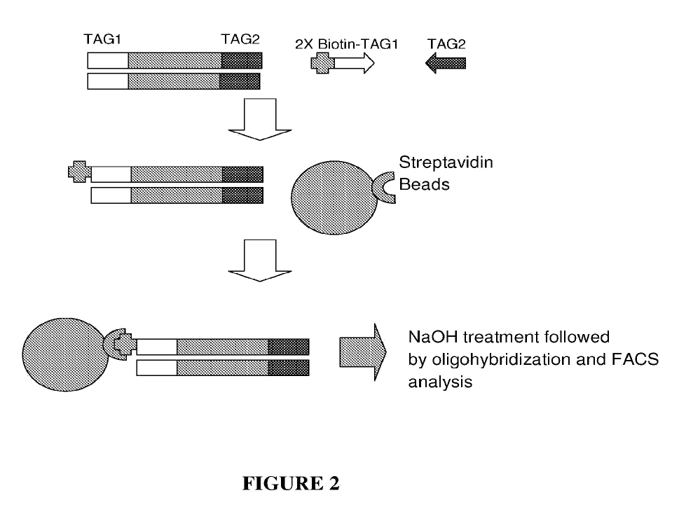

We modified the BEAMing procedure in the step of oligohybridization efficiency assessment. As shown in FIG. 2, we diluted the amplicons from the pre-amplification PCR and used the diluted amplicon as a template in a second standard PCR reaction in which the forward primer was now the 5′-dual biotin-PEGspacer18-t-TAG1 oligonucleotide. The resulting amplicons from the second PCR had biotin tags. These tagged amplicons were then incubated with streptavidin beads, which were then washed with sodium hydroxide to liberate the non-biotinylated strand and used for assaying for oligohybridization efficiency using the fluorescence activated cell sorting method (FACS). FACS is known in the art to have the capability of sorting cells/beads according to the fluorescence signals on the cells/beads (Lo et al., 2008).

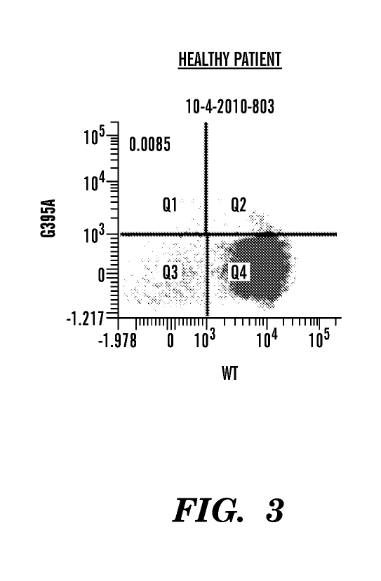

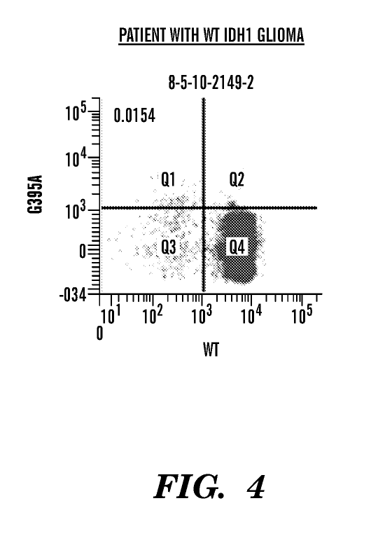

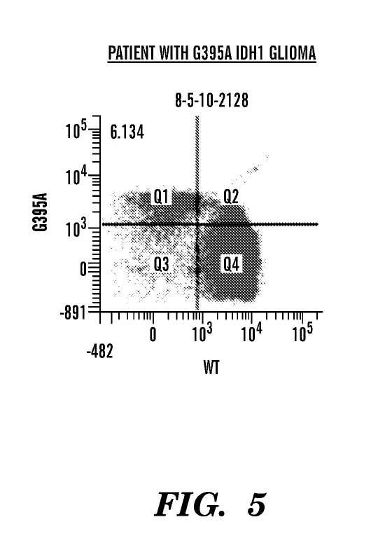

Using the modified BEAMing PCR method, we analyzed IDH1 gene in each of the nucleic acid extractions from microvesicles isolated from healthy individuals, glioma patients with wide-type IDH1, and glioma patients with G395A mutant IDH1. As shown in FIG. 3, the amplicon signals from the healthy individuals were mostly confined to the Q4 region, suggesting the IDH1 amplicons were wild-type IDH1. As shown in FIG. 4, the amplicon signals from the glioma patients with wide-type IDH1 were also mostly confined to the Q4 region, suggesting the IDH1 amplicons were wild-type IDH1. As shown in FIG. 5, the amplicon signals from the glioma patients with G395A mutant IDH1 were located in both the Q4 and Q1 regions, suggesting the G395A mutant IDH1 amplicons were present.

TABLE 2
IDH1 gene assay results. The pathology and IDH1 status of each sample
was based on the clinical and conventional pathological examination
of each tumor biopsy. The BEAMing Analysis Results were obtained
through the BEAMing PCR analysis of nucleic acids extracted from
microvesicles isolated from patient serum samples.

| | IDH1 | BEAMing Analysis |
|---|---|---|
| Sample and its Pathology | Status | Results |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT | WT |
| Healthy | WT(?) | R132H* |
| Anaplastic astrocytoma, WHO grade III | WT | WT |
| Anaplastic astrocytoma, WHO grade III | WT | WT |
| Diffuse astrocytoma, WHO Grade II | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | R132H* |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | R132H* |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma, WHO grade IV | WT | WT |
| Glioblastoma (WHO grade IV) with giant | WT | WT |
| cell features | | |
| Recurrent/residual glioblastoma (WHO | WT | WT |
| grade IV) | | |
| Glioma, consistent w/astrocytoma, WHO | WT | WT |
| Grade II | | |
| Glioma with vascular proliferation | WT | WT |
| High Grade Glioma w/extensive necrosis | WT | WT |
| High Grade Glioma w/extensive necrosis | WT | WT |
| Dysplastic Gangliocytoma | WT | WT |
| Metastatic adenocarcinoma | WT | WT |
| Anaplastic astrocytoma, WHO Grade III | R132H | WT |
| Anaplastic astrocytoma, WHO grade III, | R132H | WT |
| with prominent giant cell component | | |
| Anaplastic oligodendroglioma, WHO | R132H | R132H |
| grade III | | (retested to be WT) |
| Anaplastic oligodendrogiioma WHO | R132H | WT |
| Grade III | | |
| Anaplastic oligodendroglioma, WHO | R132H | WT |
| grade III | | |
| Anaplastic oligodendroglioma, WHO | R132H | WT |
| grade III | | |
| Glioma compatible with GBM, WHO | R132H | WT |
| Grade IV | | |
| Oligoastrocytoma, WHO Grade II | R132H | WT |
| Oligoastrocytoma, WHO grade III | R132C | WT |
| Oligoastrocytoma, WHO grade III | R132H | WT |
| Oligoastrocytoma WHO grade III | R132H | WT |
| Oligoastrocytoma, WHO grade III | R132H | R132H |
| | | (retested to be WT) |
| unknown | WT | WT |
| unknown | WT | WT |
| unknown | WT | WT |
| unknown | R132H | WT |

As shown in Table 2, using the microvesicle-based assay method disclosed herein, we analyzed 20 samples from healthy individuals, 36 samples from patients with wild-type IDH1, 12 samples from patients with mutant IDH1, and 4 samples from individuals without known pathological status. Among the 20 samples from healthy individuals, 19 samples were identified to have wild-type IDH. Among the 36 samples from patients with wild-type IDH1, 34 samples were identified to have wild-type IDH1. Among the 12 samples from patients with mutant IDH1, 2 samples were initially tested to have mutant IDH1 but later retested to have wild-type IDH1. The 4 samples without known pathological status were tested to have wild-type IDH1.

With the BEAMing PCR results from all 29 glioma patients, we found that this BEAMing PCR technology had a mutation detection limit of about 0.1%, i.e., one G395A mutant in 1000 wild-type IDH1 copies.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

### REFERENCES

Abravaya, K., J. J. Carrino, S. Muldoon, and H. H. Lee. 1995. Detection of point mutations with a modified ligase chain reaction (Gap-LCR). Nucleic Acids Res. 23:675-82.
Al-Nedawi, K., B. Meehan, J. Micallef, V. Lhotak, L. May, A. Guha, and J. Rak. 2008. Intercellular transfer of the oncogenic receptor EGFRvIII by microvesicles derived from tumour cells. Nat Cell Biol. 10:619-24.
Bossi, A., F. Bonini, A. P. Turner, and S. A. Piletsky. 2007. Molecularly imprinted polymers for the recognition of proteins: the state of the art. Biosens Bioelectron. 22:1131-7.
Cheruvanky, A., H. Zhou, T. Pisitkun, J. B. Kopp, M. A. Knepper, P. S. Yuen, and R. A. Star. 2007. Rapid isolation of urinary exosomal biomarkers using a nanomembrane ultrafiltration concentrator. Am J Physiol Renal Physiol. 292:F1657-61.
Cotton, R. G., N. R. Rodrigues, and R. D. Campbell. 1988. Reactivity of cytosine and thymine in single-base-pair mismatches with hydroxylamine and osmium tetroxide and its application to the study of mutations. Proc Natl Acad Sci USA. 85:4397-401.
Diehl, F., M. Li, Y. He, K. W. Kinzler, B. Vogelstein, and D. Dressman. 2006. BEAMing: single-molecule PCR on microparticles in water-in-oil emulsions. Nat Methods. 3:551-9.
Diehl, F., K. Schmidt, M. A. Choti, K. Romans, S. Goodman, M. Li, K. Thornton, N. Agrawal, L. Sokoll, S. A. Szabo, K. W. Kinzler, B. Vogelstein, and L. A. Diaz, Jr. 2008. Circulating mutant DNA to assess tumor dynamics. Nat Med. 14:985-90.
Fiorentino, F., M. C. Magli, D. Podini, A. P. Ferraretti, A. Nuccitelli, N. Vitale, M. Baldi, and L. Gianaroli. 2003. The minisequencing method: an alternative strategy for preimplantation genetic diagnosis of single gene disorders. Mol Hum Reprod. 9:399-410.
Fischer, S. G., and L. S. Lerman. 1979a. Length-independent separation of DNA restriction fragments in two-dimensional gel electrophoresis. Cell. 16:191-200.
Fischer, S. G., and L. S. Lerman. 1979b. Two-dimensional electrophoretic separation of restriction enzyme fragments of DNA. Methods Enzymol. 68:183-91.
Furnari, F. B., T. Fenton, R. M. Bachoo, A. Mukasa, J. M. Stommel, A. Stegh, W. C. Hahn, K. L. Ligon, D. N. Louis, C. Brennan, L. Chin, R. A. DePinho, and W. K. Cavenee. 2007. Malignant astrocytic glioma: genetics, biology, and paths to treatment. Genes Dev. 21:2683-710.
Geiss, G. K., R. E. Bumgarner, B. Birditt, T. Dahl, N. Dowidar, D. L. Dunaway, H. P. Fell, S. Ferree, R. D. George, T. Grogan, J. J. James, M. Maysuria, J. D. Mitton, P. Oliveri, J. L. Osborn, T. Peng, A. L. Ratcliffe, P. J. Webster, E. H. Davidson, and L. Hood. 2008. Direct multiplexed measurement of gene expression with color-coded probe pairs. Nat Biotechnol. 26:317-25.
Gormally, E., E. Caboux, P. Vineis, and P. Hainaut. 2007. Circulating free DNA in plasma or serum as biomarker of carcinogenesis: practical aspects and biological significance. Mutat Res. 635:105-17.
Green, C. L., C. M. Evans, R. K. Hills, A. K. Burnett, D. C. Linch, and R. E. Gale. 2010. The prognostic significance of IDH1 mutations in younger adult patients with acute myeloid leukemia is dependent on FLT3/ITD status. Blood. 116:2779-82.
Guatelli, J. C., K. M. Whitfield, D. Y. Kwoh, K. J. Barringer, D. D. Richman, and T. R. Gingeras. 1990. Isothermal, in vitro amplification of nucleic acids by a multienzyme reaction modeled after retroviral replication. Proc Natl Acad Sci USA. 87:1874-8.
Hartmann, C., B. Hentschel, W. Wick, D. Capper, J. Felsberg, M. Simon, M. Westphal, G. Schackert, R. Meyermann, T. Pietsch, G. Reifenberger, M. Weller, M. Loeffler, and A. von Deimling. 2010. Patients with IDH1 wild type anaplastic astrocytomas exhibit worse prognosis than IDH1-mutated glioblastomas, and IDH1 mutation status accounts for the unfavorable prognostic effect of higher age: implications for classification of gliomas. Acta Neuropathol. 120:707-18.
Johnson, S., D. Evans, S. Laurenson, D. Paul, A. G. Davies, P. K. Ferrigno, and C. Wald. 2008. Surface-immobilized peptide aptamers as probe molecules for protein detection. Anal Chem. 80:978-83.
Jones, S., X. Zhang, D. W. Parsons, J. C. Lin, R. J. Leary, P. Angenendt, P. Mankoo, H. Carter, H. Kamiyama, A. Jimeno, S. M. Hong, B. Fu, M. T. Lin, E. S. Calhoun, M. Kamiyama, K. Walter, T. Nikolskaya, Y. Nikolsky, J. Hartigan, D. R. Smith, M. Hidalgo, S. D. Leach, A. P. Klein, E. M. Jaffee, M. Goggins, A. Maitra, C. Iacobuzio-Donahue, J. R.
Eshleman, S. E. Kern, R. H. Hruban, R. Karchin, N. Papadopoulos, G. Parmigiani, B. Vogelstein, V. E. Velculescu, and K. W. Kinzler. 2008. Core Signaling Pathways in Human Pancreatic Cancers Revealed by Global Genomic Analyses. Science.
Kan, Y. W., and A. M. Dozy. 1978a. Antenatal diagnosis of sickle-cell anaemia by D. N. A. analysis of amniotic-fluid cells. Lancet. 2:910-2.
Kan, Y. W., and A. M. Dozy. 1978b. Polymorphism of DNA sequence adjacent to human beta-globin structural gene: relationship to sickle mutation. Proc Natl Acad Sci USA. 75:5631-5.
Kwoh, D. Y., G. R. Davis, K. M. Whitfield, H. L. Chappelle, L. J. DiMichele, and T. R. Gingeras. 1989. Transcription-based amplification system and detection of amplified human immunodeficiency virus type 1 with a bead-based sandwich hybridization format. Proc Natl Acad Sci USA. 86:1173-7.
Landegren, U., R. Kaiser, J. Sanders, and L. Hood. 1988. A ligase-mediated gene detection technique. Science. 241:1077-80.
Li, J., L. Wang, H. Mamon, M. H. Kulke, R. Berbeco, and G. M. Makrigiorgos. 2008. Replacing PCR with COLD-PCR enriches variant DNA sequences and redefines the sensitivity of genetic testing. Nat Med. 14:579-84.
Liu, Q., J. C. Greimann, and C. D. Lima. 2006. Reconstitution, activities, and structure of the eukaryotic RNA exosome. Cell. 127:1223-37.
Lo, K., R. R. Brinkman, and R. Gottardo. 2008. Automated gating of flow cytometry data via robust model-based clustering. Cytometry A. 73:321-32.
McLendon, R., A. Friedman, D. Bigner, E. G. Van Meir, D. J. Brat, G. Marie Mastrogianakis, J. J. Olson, T. Mikkelsen, N. Lehman, K. Aldape, W. K. Alfred Yung, O. Bogler, S. Vandenberg, M. Berger, M. Prados, D. Muzny, M. Morgan, S. Scherer, A. Sabo, L. Nazareth, L. Lewis, O. Hall, Y. Zhu, Y. Ren, O. Alvi, J. Yao, A. Hawes, S. Jhangiani, G. Fowler, A. San Lucas, C. Kovar, A. Cree, H. Dinh, J. Santibanez, V. Joshi, M. L. Gonzalez-Garay, C. A. Miller, A. Milosavljevic, L. Donehower, D. A. Wheeler, R. A. Gibbs, K. Cibulskis, C. Sougnez, T. Fennell, S. Mahan, J. Wilkinson, L. Ziaugra, R. Onofrio, T. Bloom, R. Nicol, K. Ardlie, J. Baldwin, S. Gabriel, E. S. Lander, L. Ding, R. S. Fulton, M. D. McLellan, J. Wallis, D. E. Larson, X. Shi, R. Abbott, L. Fulton, K. Chen, D. C. Koboldt, M. C. Wendl, R. Meyer, Y. Tang, L. Lin, J. R. Osborne, B. H. Dunford-Shore, T. L. Miner, K. Delehaunty, C. Markovic, G. Swift, W. Courtney, C. Pohl, S. Abbott, A. Hawkins, S. Leong, C. Haipek, H. Schmidt, M. Wiechert, T. Vickery, S. Scott, D. J. Dooling, A. Chinwalla, G. M. Weinstock, E. R. Mardis, R. K. Wilson, G. Getz, W. Winckler, R. G. Verhaak, M. S. Lawrence, M. O'Kelly, J. Robinson, G. Alexe, R. Beroukhim, S. Carter, D. Chiang, J. Gould, et al. 2008. Comprehensive genomic characterization defines human glioblastoma genes and core pathways. Nature.
Miele, E. A., D. R. Mills, and F. R. Kramer. 1983. Autocatalytic replication of a recombinant RNA. J Mol Biol. 171:281-95.
Myers, R. M., Z. Larin, and T. Maniatis. 1985. Detection of single base substitutions by ribonuclease cleavage at mismatches in RNA:DNA duplexes. Science. 230:1242-6.
Nagrath, S., L. V. Sequist, S. Maheswaran, D. W. Bell, D. Irimia, L. Ulkus, M. R. Smith, E. L. Kwak, S. Digumarthy, A. Muzikansky, P. Ryan, U. J. Balis, R. G. Tompkins, D. A. Haber, and M. Toner. 2007. Isolation of rare circulating tumour cells in cancer patients by microchip technology. Nature. 450:1235-9.
Nakazawa, H., D. English, P. L. Randell, K. Nakazawa, N. Martel, B. K. Armstrong, and H. Yamasaki. 1994. UV and skin cancer: specific p53 gene mutation in normal skin as a biologically relevant exposure measurement. Proc Natl Acad Sci USA. 91:360-4.
Orita, M., H. Iwahana, H. Kanazawa, K. Hayashi, and T. Sekiya. 1989. Detection of polymorphisms of human DNA by gel electrophoresis as single-strand conformation polymorphisms. Proc Natl Acad Sci USA. 86:2766-70.
Parsons, D. W., S. Jones, X. Zhang, J. C. Lin, R. J. Leary, P. Angenendt, P. Mankoo, H. Carter, I. M. Siu, G. L. Gallia, A. Olivi, R. McLendon, B. A. Rasheed, S. Keir, T. Nikolskaya, Y. Nikolsky, D. A. Busam, H. Tekleab, L. A. Diaz, Jr., J. Hartigan, D. R. Smith, R. L. Strausberg, S. K. Marie, S. M. Shinjo, H. Yan, G. J. Riggins, D. D. Bigner, R. Karchin, N. Papadopoulos, G. Parmigiani, B. Vogelstein, V. E. Velculescu, and K. W. Kinzler. 2008. An Integrated Genomic Analysis of Human Glioblastoma Multiforme. Science.
Raposo, G., H. W. Nijman, W. Stoorvogel, R. Liejendekker, C. V. Harding, C. J. Melief, and H. J. Geuze. 1996. B lymphocytes secrete antigen-presenting vesicles. J Exp Med. 183:1161-72.
Skog, J., T. Wurdinger, S. van Rijn, D. H. Meijer, L. Gainche, M. Sena-Esteves, W. T. Curry, Jr., B. S. Carter, A. M. Krichevsky, and X. O. Breakefield. 2008. Glioblastoma microvesicles transport RNA and proteins that promote tumour growth and provide diagnostic biomarkers. Nat Cell Biol. 10:1470-6.
Steemers, F. J., W. Chang, G. Lee, D. L. Barker, R. Shen, and K. L. Gunderson. 2006. Whole-genome genotyping with the single-base extension assay. Nat Methods. 3:31-3.
Taylor, D. D., and C. Gercel-Taylor. 2008. MicroRNA signatures of tumor-derived exosomes as diagnostic biomarkers of ovarian cancer. Gynecol Oncol. 110:13-21.
van Dijk, E. L., G. Schilders, and G. J. Pruijn. 2007. Human cell growth requires a functional cytoplasmic exosome, which is involved in various mRNA decay pathways. RNA. 13:1027-35.
Wood, L. D., D. W. Parsons, S. Jones, J. Lin, T. Sjoblom, R. J. Leary, D. Shen, S. M. Boca, T. Barber, J. Ptak, N. Silliman, S. Szabo, Z. Derso, V. Ustyanksky, T. Nikolskaya, Y. Nikolsky, R. Karchin, P. A. Wilson, J. S. Kaminker, Z. Zhang, R. Croshaw, J. Willis, D. Dawson, M. Shipitsin, J. K. Willson, S. Sukumar, K. Polyak, B. H. Park, C. L. Pethiyagoda, P. V. Pant, D. G. Ballinger, A. B. Sparks, J. Hartigan, D. R. Smith, E. Suh, N. Papadopoulos, P. Buckhaults, S. D. Markowitz, G. Parmigiani, K. W. Kinzler, V. E. Velculescu, and B. Vogelstein. 2007. The genomic landscapes of human breast and colorectal cancers. Science. 318:1108-13.

1. A method for assaying a biological sample, comprising the steps of:
a. isolating, obtaining or using a microvesicle fraction from a biological sample; and
b. detecting in the microvesicle fraction the presence or absence of a genetic aberration in a gene selected from the group consisting of IDH1, IDH2, TP53, PTEN, CDKN2A, NF1, EGFR, RB1, PIK3CA, and BRAF.

2.-38. (canceled)


SEQUENCE LISTING (11 sequences)

SEQ ID NO: 1 - DNA, 131 bp, Homo sapiens
cggtcttcagagaagccattatctgcaaaaatatcccccggcttgtgagtggatgggtaaaacctatcatcataggtcgtcatgcttatggggatcaatacagagcaactgattttgttgttcctgggcct

SEQ ID NO: 2 - DNA, 40 bp, Artificial Sequence
  Description of Artificial Sequence Synthetic primer
tcccgcgaaattaatacgaccggtcttcagagaagccatt

SEQ ID NO: 3 - DNA, 38 bp, Artificial Sequence
  Description of Artificial Sequence Synthetic primer
gctggagctctgcagctaaggcccaggaacaacaaaat

SEQ ID NO: 4 - DNA, 21 bp, Artificial Sequence
  Description of Artificial Sequence Synthetic primer
ttcccgcgaaattaatacgac

SEQ ID NO: 5 - DNA, 20 bp, Artificial Sequence
  Description of Artificial Sequence Synthetic primer
tcccgcgaaattaatacgac

SEQ ID NO: 6 - DNA, 23 bp, Artificial Sequence
  Description of Artificial Sequence Synthetic primer
aatcagttgctctgtattgatcc

SEQ ID NO: 7 - DNA, 24 bp, Artificial Sequence
  Description of Artificial Sequence Synthetic primer
atcatcataggtcgtcatgcttat

SEQ ID NO: 8 - DNA, 20 bp, Artificial Sequence
  Description of Artificial Sequence Synthetic primer
ttgtgagtggatgggtaaaa

SEQ ID NO: 9 - DNA, 21 bp, Artificial Sequence
  Description of Artificial Sequence Synthetic primer
ataagcatgacgacctatgat

SEQ ID NO: 10 - DNA, 21 bp, Artificial Sequence
  Description of Artificial Sequence Synthetic primer
ataagcatgatgacctatgat

SEQ ID NO: 11 - DNA, 20 bp, Artificial Sequence
  Description of Artificial Sequence Synthetic primer
ttttacccatccactcacaa